(19) United States
(12) Patent Application Publication
(10) Pub. No.: US 2019/0099756 A1
(43) Pub. Date: Apr. 4, 2019

(54) MICRO-FLUIDIC CHIP, DRIVING METHOD THEREOF, MICRO-FLUIDIC ELEMENT AND BIOSENSOR

(71) Applicants: BOE TECHNOLOGY GROUP CO., LTD., Beijing (CN); BEIJING BOE OPTOELECTRONICS TECHNOLOGY CO., LTD., Beijing (CN)

(72) Inventors: Fengchun Pang, Beijing (CN); Peizhi Cai, Beijing (CN); Yue Geng, Beijing (CN); Le Gu, Beijing (CN); Chuncheng Che, Beijing (CN)

(73) Assignees: BOE TECHNOLOGY GROUP CO., LTD., Beijing (CN); BEIJING BOE OPTOELECTRONICS TECHNOLOGY CO., LTD., Beijing (CN)

(21) Appl. No.: 15/922,084

(22) Filed: Mar. 15, 2018

(30) Foreign Application Priority Data
Sep. 29, 2017 (CN) 201710912397.5

(51) Int. Cl. B01L 3/00; B01L 9/00; B01F 13/00; G01N 33/543

(52) U.S. Cl. CPC B01L 3/502792; B01L 3/50273; B01L 3/502746; B01L 3/502753; B01L 2300/0636; B01L 9/527; B01F 13/0071; G01N 33/5438; B01L 3/502715

(57) ABSTRACT

The present disclosure provides a micro-fluidic chip, a driving method thereof, a micro-fluidic element and a biosensor. The micro-fluidic chip includes a photoelectric conversion layer, a first electrode, and a second electrode arranged opposite to the first electrode. A channel for droplets is arranged between the first electrode and the photoelectric conversion layer. The photoelectric conversion layer is arranged at a side of the second electrode adjacent to the first electrode and configured to convert an incident light beam into a charge signal to drive the droplets in the channel to move.

### CROSS-REFERENCE TO RELATED APPLICATION

The present application claims a priority of the Chinese Patent Application No. 201710912397.5 filed on Sep. 29, 2017, which is incorporated herein by reference in its entirety.

### TECHNICAL FIELD

The present disclosure relates to the field of micro-fluidic technology, in particular to a micro-fluidic chip, a driving method thereof, a micro-fluidic element and a biosensor.

### BACKGROUND

Digital micro-fluidic technique is used for operating discrete droplets, and it includes two parts, i.e., generation of the droplets and operation of the droplets. The generation of the droplets includes generating a small amount of droplets at a nanometer scale or micrometer scale, and this procedure takes a very short time period. The operation of the droplets includes such basic treatment as generation, transportation, mixing and separation. The digital micro-fluidic technique may be used to operate a plurality of droplets simultaneously, to perform parallel processing, detection and analysis on the droplets in a lab-on-chip, thereby to remarkably improve the working efficiency.

Through the digital micro-fluidic technique, it is able to integrate such basic operational units for sample preparation, reaction, separation and detection during the biological, chemical or medical analysis into a chip at a micrometer scale, to automatically complete the entire analysis. The digital micro-fluidic technique has shown a great prospect in biological, chemical and medical fields due to such advantages as low cost, short detection time period and high sensitivity.

Recently, an electrowetting-on-dielectric (EWOD)-based digital micro-fluidic technique has been used for operate the discrete droplets, and it has become a research hotspot in the industry due to such advantages as small reagent consumption, low manufacture cost, being free of cross contamination, being capable of operate a single droplet, and being capable of used for a portable system.

However, for a current micro-fluidic chip, it is necessary to connect a driving electrode to an external circuit, so an additional step of forming an electrode wire is required. In addition, the design of the driving circuit is complex, resulting in high manufacture cost.

### SUMMARY

In one aspect, the present disclosure provides in some embodiments a micro-fluidic chip, including a photoelectric conversion layer, a first electrode, and a second electrode arranged opposite to the first electrode. A channel for droplets is arranged between the first electrode and the photoelectric conversion layer. The photoelectric conversion layer is arranged at a side of the second electrode adjacent to the first electrode and configured to convert an incident light beam into a charge signal, to drive the droplets in the channel to move.

In a possible embodiment of the present disclosure, the photoelectric conversion layer is a Positive-Intrinsic-Negative (PIN) photoelectric semiconductor layer.

In a possible embodiment of the present disclosure, the PIN photoelectric semiconductor layer includes a P-type semiconductor layer, an I-type semiconductor layer and an N-type semiconductor layer laminated one on another, and the N-type semiconductor layer is arranged at a side of the second electrode adjacent to the first electrode.

In a possible embodiment of the present disclosure, the P-type semiconductor layer is a P-type amorphous silicon (a-Si) layer, and the I-type semiconductor layer is an I-type a-Si layer, and the N-type semiconductor layer is an N-type a-Si layer.

In a possible embodiment of the present disclosure, the micro-fluidic chip further includes a dielectric layer arranged at a side of the photoelectric conversion layer adjacent to the first electrode, and the channel is arranged between the first electrode and the dielectric layer.

In a possible embodiment of the present disclosure, the dielectric layer is made of at least one of silicon nitride, silicon dioxide and ferroelectric copolymer.

In a possible embodiment of the present disclosure, the micro-fluidic chip further includes a first hydrophobic layer arranged at a side of the first electrode adjacent to the second electrode and a second hydrophobic layer arranged at a side of the dielectric layer adjacent to the first electrode, and the channel is arranged between the first hydrophobic layer and the second hydrophobic layer.

In a possible embodiment of the present disclosure, the micro-fluidic chip further includes a third electrode arranged between the dielectric layer and the photoelectric conversion layer.

In a possible embodiment of the present disclosure, the third electrode is an electrode array.

In a possible embodiment of the present disclosure, the first electrode is a surface-like electrode (planar electrode), and/or the second electrode is a surface-like electrode.

In another aspect, the present disclosure provides in some embodiments a method for driving a micro-fluidic chip, including steps of: applying a voltage between a first electrode and a second electrode; and converting, by a photoelectric conversion layer, an incident light beam into a charge signal, to drive droplets in a channel to move.

In yet another aspect, the present disclosure provides in some embodiments a micro-fluidic element including the above-mentioned micro-fluidic chip.

In still yet another aspect, the present disclosure provides in some embodiments a biosensor including the above-mentioned micro-fluidic element.

### BRIEF DESCRIPTION OF THE DRAWINGS

FIG. 1 is a schematic view showing a micro-fluidic chip in the case that no electric field is applied;

FIG. 2 is a schematic view showing the micro-fluidic chip in the case that an electric field is applied;

FIG. 3 is a schematic view showing a micro-fluidic chip according to one embodiment of the present disclosure;

FIG. 4 is a schematic view showing the micro-fluidic chip irradiated by light according to one embodiment of the present disclosure;

FIG. 5 is another schematic view showing the micro-fluidic chip according to one embodiment of the present disclosure;

FIG. 6 is yet another schematic view showing the micro-fluidic chip according to one embodiment of the present disclosure;

FIG. 7 is still yet another schematic view showing the micro-fluidic chip according to one embodiment of the present disclosure;

FIG. 8 is still yet another schematic view showing the micro-fluidic chip according to one embodiment of the present disclosure;

FIG. 9 is another schematic view showing the micro-fluidic chip in the case of being irradiated by light according to one embodiment of the present disclosure; and

FIG. 10 is a schematic view showing the micro-fluidic chip where a droplet is driven by the light to move according to one embodiment of the present disclosure.

### DETAILED DESCRIPTION OF THE EMBODIMENTS

In order to make the objects, the technical solutions and the advantages of the present disclosure more apparent, the present disclosure will be described hereinafter in a clear and complete manner in conjunction with the drawings and embodiments.

Unless otherwise defined, any technical or scientific term used herein shall have the common meaning understood by a person of ordinary skills. Such words as “first” and “second” used in the specification and claims are merely used to differentiate different components rather than to represent any order, number or importance. Similarly, such words as “one” or “one of” are merely used to represent the existence of at least one member, rather than to limit the number thereof. Such words as “connect” or “connected to” may include electrical connection, direct or indirect, rather than to be limited to physical or mechanical connection. Such words as “on”, “under”, “left” and “right” are merely used to represent relative position relationship, and when an absolute position of the object is changed, the relative position relationship will be changed too.

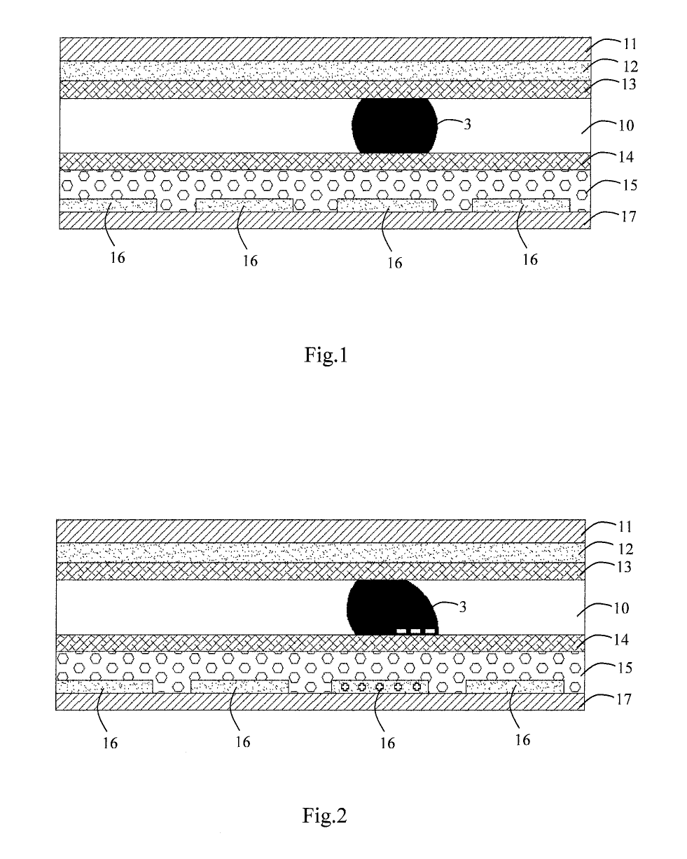

An EWOD-based digital micro-fluidic technique is mainly adopted by a digital micro-fluidic chip. As shown in FIG. 1, the digital micro-fluidic chip is of a biplanar sandwich structure, and it includes an upper substrate 11, a lower substrate 17, and an upper electrode 12 and a lower electrode 16 arranged between the upper substrate 11 and the lower substrate 17. The upper electrode 12 is a surface-layer electrode, and the lower electrode 16 is an electrode capable of being controlled individually or an electrode array. A dielectric layer 15 is further arranged on the lower electrode 16. In addition, an upper hydrophobic layer 13 is coated onto the upper electrode 12 by spinning, and a lower hydrophobic layer 14 is coated onto the dielectric layer 15 by spinning A channel 10 for droplets 3 is arranged between the upper hydrophobic layer 13 and the lower hydrophobic layer 14. The droplets are sandwiched between two plate-like electrodes. The upper electrode 12 serves as a ground electrode as a whole, and the lower electrode 16 may include a plurality of micro-electrode arrays capable of being control individually.

In the case that no external electric field is applied, the droplet is in a hydrophobic state and keeps still, as shown in FIG. 1. In the case that a gravity is omitted, a contact angle θ(0) of the droplet on a solid surface may be expressed in the following Young's equation:

$$cos θ ( 0 ) = \frac{{γ}_{sol \text{-} gas} - {γ}_{sol \text{-} liq}}{{γ}_{gas \text{-} liq}} ,$$

where γ_{sol-gas}, γ_{sol-liq} and γ_{gas-liq} represent surface tension coefficients between a solid and a gas, between a solid and a liquid and between a gas and a liquid respectively.

In the case that a positive voltage V is applied to the lower electrode 16 and the upper electrode 12 is grounded, an electric field is generated between the upper substrate 11 and the lower substrate 17, so a lower-right part of the droplet is changed from the hydrophobic state to a hydrophilic state, and thereby the droplet moves to the right, as shown in FIG. 2.

At this time, a contact angle θ(V) of the droplet at the solid surface is expressed in the following Young-Lippmann's equation:

$$cos θ ( V ) = cos θ ( 0 ) + \frac{{ɛ}_{0} {ɛ}_{r}}{2 d {γ}_{gas \text{-} liq}} {V}^{2} ,$$

where εᵣ represents a relative dielectric constant of the dielectric layer, ε₀ represents an absolute dielectric constant in vacuum, and d represents a thickness of the dielectric layer. Based on the Young-Lippmann's equation, a change in the contact angle is related to an external potential. The change in the contact angle may increase along with an increase in the external potential.

Hence, it is necessary to connect driving electrodes (i.e., the lower electrodes) of the micro-fluidic chip to an external circuit, to drive different driving electrodes, thereby to drive the droplets to move. At this time, an additional step of forming an electrode wire is required. In addition, the design of the driving circuit is complex, resulting in high manufacture cost.

An object of the present disclosure is to provide a micro-fluidic chip, a driving method thereof, a micro-fluidic element and a biosensor, to solve the above-mentioned problem.

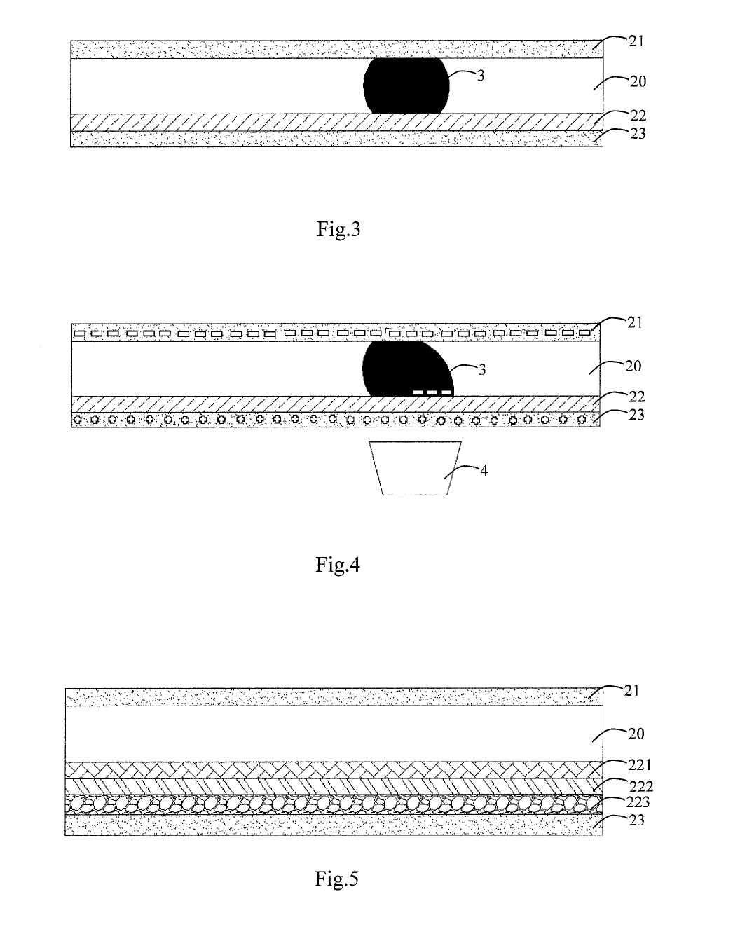

The present disclosure provides in some embodiments a micro-fluidic chip which, as shown in FIG. 3, includes a photoelectric conversion layer 22, a first electrode 21, and a second electrode 23 arranged opposite to the first electrode 21. A channel 20 for droplets 3 is arranged between the first electrode 21 and the photoelectric conversion layer 22. The photoelectric conversion layer 22 is arranged at a side of the second electrode 23 adjacent to the first electrode 21 and configured to convert an incident light beam into a charge signal, to drive the droplets in the channel 20 to move.

As shown in FIG. 4, the droplet 3 is placed into the channel 20, and a voltage is applied between the first electrode 21 and the second electrode 23. An incident light beam 4 is converted by the photoelectric conversion layer 22, into a charge signal, thereby to drive the droplet 3 in the channel 20 to move. To be specific, the photoelectric conversion layer 22 is irradiated by the incident light beam 4, so as to generate a large amount of negative and positive charges. The negative charges move toward the second electrode 23, and the positive charges move toward the first electrode 21, so as to generate an electric field between the first electrode 21 and the photoelectric conversion layer 22. In the case that a right part of the droplet 3 is irradiated by the incident light beam 4, a large amount of negative charges may be generated at a lower-right surface of the droplet 3 (i.e., a side of the droplet 3 adjacent to the second electrode). At this time, a surface tension at the lower-right side of the droplet 3 may be changed, i.e., a lower-right part of the droplet 3 may be changed from a hydrophobic state to a hydrophilic state, so as to drive the droplet 3 to move to the right. Identically, in the case that a lower-left side of the droplet 3 (i.e., a side of the droplet 3 adjacent to the second electrode) is irradiated by the incident light beam 4, a lower-left part of the droplet 3 may be changed from the hydrophobic state to the hydrophilic state, so as to drive the droplet 3 to the left.

It should be appreciated that, in the embodiments of the present disclosure, the first electrode 21 and the second electrode 23 may each be a surface-like (planar) electrode, and the incident light beam may reach different positions of the photoelectric conversion layer 22, to drive the droplet to move. In addition, the second electrode 23 is a transparent electrode, to prevent the second electrode 23 from shielding the incident light beam. In a possible embodiment of the present disclosure, the first electrode 21 and/or the second electrode 23 may be a transparent conductive film made of indium tin oxide (ITO). The incident light beam 4 may be a laser beam, so as to accurately control an irradiation position of the incident light beam 4, thereby to accurately control the movement of the droplet.

According to the micro-fluidic chip in the embodiments of the present disclosure, the external electric field is applied between the first electrode and the second electrode, and the photoelectric conversion layer is irradiated by the incident light beam, so as to generate the negative and positive charges, generate the electric field between the photoelectric conversion layer and the first electrode, and change the hydrophobic state of the droplet to the hydrophilic state, thereby to drive the droplet to move. The droplet is driven to move by the incident light beam, so it is unnecessary to connect the driving electrode to an external circuit and provide a wire therebetween, thereby it is able to facilitate the design of a large-scale circuit and the manufacture of a large-size digital micro-fluidic chip, and prevent the droplet from being adversely affected by the electrode wire. In addition, it is merely necessary to apply a negative voltage and a positive voltage to the first electrode and the second electrode respectively, so it is unnecessary to provide a complex circuit driving design and a high-voltage power source, thereby to effectively reduce the manufacture coast and simplify the manufacture process.

In a possible embodiment of the present disclosure, the photoelectric conversion layer 22 may be a PIN photoelectric semiconductor layer. In the case that the PIN photoelectric semiconductor layer 22 is irradiated by the incident light beam 4, photoelectrons and holes may be generated in the PIN photoelectric semiconductor layer. In addition, under the effect of a backward bias voltage, the photoelectrons may move toward the second electrode 23, and the holes may move toward the first electrode 21, so as to generate the electric field between the first electrode 21 and the photoelectric conversion layer 22, and change a surface of the droplet irradiated by the incident light beam from the hydrophobic state to the hydrophilic state, thereby to drive the droplet to move.

In a possible embodiment of the present disclosure, as shown in FIG. 5, the PIN photoelectric semiconductor layer 22 includes a P-type semiconductor layer 221, an I-type semiconductor layer 222 and an N-type semiconductor layer 223 laminated one on another, and the N-type semiconductor layer 223 is arranged at a side of the second electrode 23 adjacent to the first electrode 21. In other words, the P-type semiconductor layer 221 is arranged away from the second electrode 23. Hence, in the case that the PIN photoelectric semiconductor layer 22 is irradiated by the incident light beam 4, the photoelectrons and holes may be generated in the PIN photoelectric semiconductor layer 22. In addition, under the effect of a backward bias voltage, the photoelectrons move toward the N-type semiconductor layer 223, and the holes move toward the P-type semiconductor layer 221. As a result, it is able to generate the electric field between the first electrode 21 and the P-type semiconductor layer 221, generate a large amount of negative charges at a surface of the droplet, change the surface tension of the droplet and change the droplet from the hydrophobic state to the hydrophilic state, thereby to drive the droplet to move.

In a possible embodiment of the present disclosure, the P-type semiconductor layer 221 is a P-type a-Si layer, the I-type semiconductor layer 222 is an I-type a-Si layer, and the N-type semiconductor layer 223 is an N-type a-Si layer, so as to generate a large amount of photoelectrons and holes, and enable the photoelectrons and the holes to move toward the N-type a-Si layer and the P-type a-Si layer.

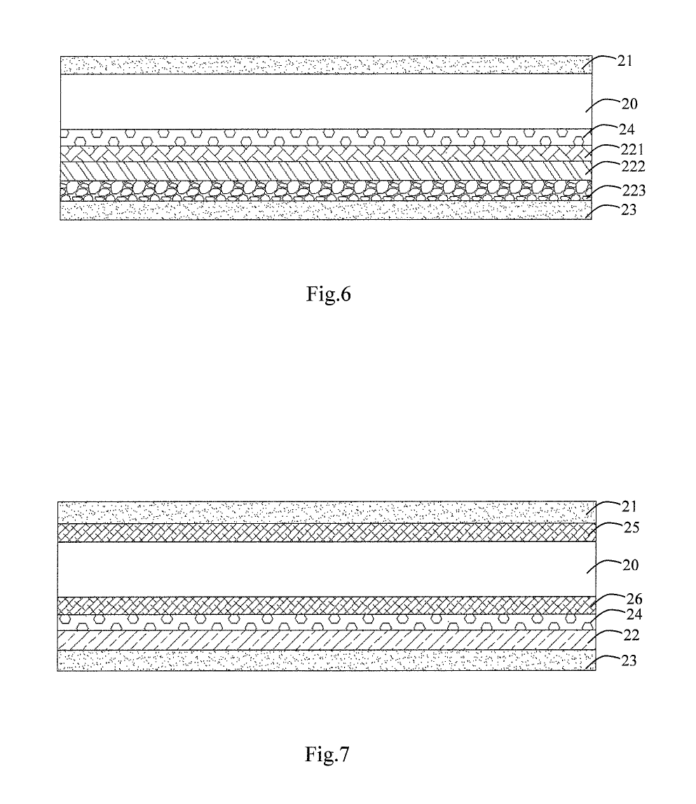

As shown in FIG. 6, in a possible embodiment of the present disclosure, the micro-fluidic chip further includes a dielectric layer 24 arranged at a side of the photoelectric conversion layer 22 adjacent to the first electrode 21, and the channel 20 is arranged between the first electrode and the dielectric layer. As a result, it is able to prevent the droplet 3 from being in direct contact with the photoelectric conversion layer 22 while ensure an electrowetting effect between the droplet and the dielectric layer, thereby to prevent the droplet from being electrolyzed. The dielectric layer is made of an insulating layer, so as to prevent the exchange of charges, thereby to re-distribute interfacial charges in the case that a high-intensity electric field is applied. Hence, it is able to acquire a relatively large change in a contact angle at an initial hydrophobic interface, thereby to acquire a relatively large driving force.

In a possible embodiment of the present disclosure, the dielectric layer may be made of at least one of silicon nitride, silicon dioxide and ferroelectric copolymer, or resin, so as to ensure the electrowetting effect between the droplet and the dielectric layer, and change the contact angle between the droplet and the dielectric layer, thereby to drive the droplet to move. The dielectric layer may also be made of Parylene C, PzT, or P(VDF-TrFE). Parylene C has excellent electrical, chemical and mechanical properties. PZT (Pb(ZrxTi(1−x))O₃) is an excellent piezoelectric, ferroelectric material, and has a very large dielectric constant. P(VDF-TrFE) is an organic high-polymer ferroelectric, piezoelectric material, and has a large dielectric constant and excellent mechanical and insulating properties. More importantly, P(VDF-TrFE) has a large light transmittance to visible light, so it is able for a detection signal from a fully-integrated silicon-based biosensor to be transmitted to a photosensitive sensor system at a bottom layer.

As shown in FIG. 7, in a possible embodiment of the present disclosure, the micro-fluidic chip further includes a first hydrophobic layer 25 arranged at a side of the first electrode 21 adjacent to the second electrode 23 and a second hydrophobic layer 26 arranged at a side of the dielectric layer 24 adjacent to the first electrode 21, and the channel 20 is arranged between the first hydrophobic layer 25 and the second hydrophobic layer 26. In this way, it is able to provide a large initial solid-liquid contact angle with a large varying range, thereby to acquire a large driving force.

In a possible embodiment of the present disclosure, the first hydrophobic layer 25 and/or the second hydrophobic layer 26 may be made of Teflon or CYTOP, so as to increase the contact angle between the droplet and the first electrode, thereby to increase the contact angle between the droplet and the dielectric layer. At this time, it is able to provide the solid-liquid contact angle with a large varying range and generate a large surface tension gradient, thereby to acquire a large driving force. In addition, it is able to reduce a contact area between the droplet and the solid surface, thereby to reduce a flow resistance to the droplet.

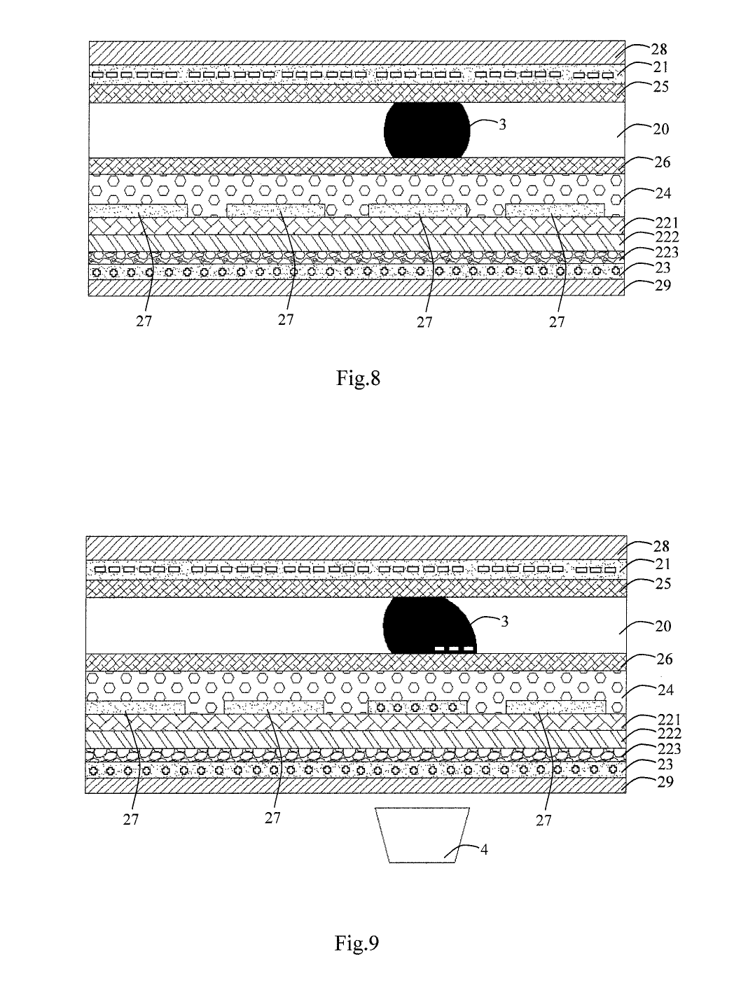

As shown in FIG. 8, in a possible embodiment of the present disclosure, the micro-fluidic chip further includes a third electrode 27 arranged between the dielectric layer 24 and the photoelectric conversion layer 22, to generate an electric field between the third electrode 27 and the droplet 3. In another possible embodiment of the present disclosure, the third electrode 27 is an electrode array, to generate the electric field merely between the electrode corresponding to the portion of the photoelectric conversion layer irradiated by the incident light beam and the first electrode, thereby to accurately control the movement of the droplet. According to the micro-fluidic chip in the embodiments of the present disclosure, it is able to accurately irradiate the portion of the photoelectric conversion layer, thereby to control the surface tension of different portions of the droplet individually. The third electrode may also be a transparent conductive layer made of ITO.

In a possible embodiment of the present disclosure, the micro-fluidic chip may further include a first substrate 28 and a second substrate 29. The first substrate 28 is arranged at a side of the first electrode 21 away from the second electrode 23, and the second substrate 29 is arranged at a side of the second electrode 23 away from the first electrode 21. The first substrate 28 and the second substrate 29 function as to support and protect the micro-fluidic chip. In another possible embodiment of the present disclosure, the second substrate 29 is a transparent substrate, so as not to shield the incident light beam from irradiating the photoelectric conversion layer 22.

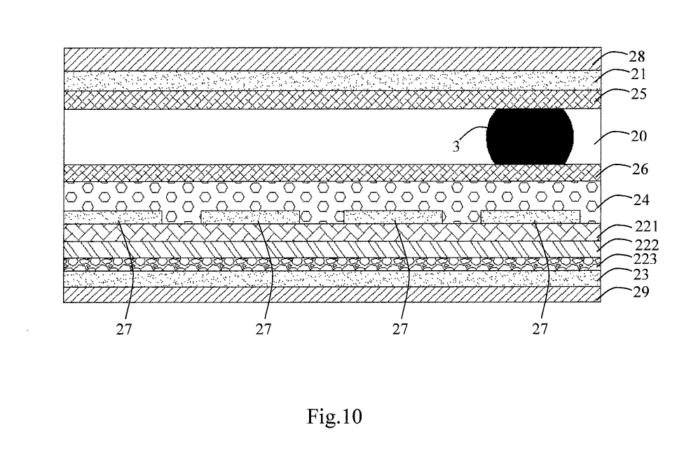

FIG. 9 shows the micro-fluidic chip irradiated by light. The negative voltage is applied to the first electrode 21, and the positive voltage is applied to the second electrode 23. In the case that the second substrate 29 is irradiated by the incident light beam 4, the incident light beam 4 may pass through the second substrate 29 and reach the PIN photoelectric semiconductor layer 22, so as to generate the photoelectrons and holes in the PIN photoelectric semiconductor layer 22. In addition, under the effect of the reverse bias voltage, the photoelectrons move to the N-type semiconductor layer 223, and the holes move to the P-type semiconductor layer 221, so as to generate the electric field between the first electrode 21 and the P-type semiconductor layer 21, generate a large amount of negative charges at a surface of the droplet, change the surface tension of the droplet, and change the droplet from the hydrophobic state to the hydrophilic state, thereby to drive the droplet to move, as shown in FIG. 10.

To be specific, in the case that a right part of the droplet is irradiated by the incident light beam, a large amount of negative charges may be generated at a lower-right part of the droplet (i.e., a side adjacent to the second electrode), so as to change the surface tension of the lower-right part of the droplet, change the lower-right part of the droplet from the hydrophobic state to the hydrophilic state, thereby to drive the droplet to the right. In the case that the lower-right part of the droplet is continuously irradiated by the incident light beam, the droplet may move to the right continuously, as shown in FIG. 10. Identically, in the case that a lower-left part of the droplet (i.e., a side adjacent to the second electrode) is irradiated by the incident light beam, the lower-left part of the droplet may be changed from the hydrophobic state to the hydrophilic state, so as to drive the droplet to move to the left.

The present disclosure further provides in some embodiments a method for driving a micro-fluidic chip, including steps of: applying a voltage between a first electrode and a second electrode; and converting, by a photoelectric conversion layer, an incident light beam into a charge signal, so as to drive droplets in a channel to move.

To be specific, the photoelectric conversion layer is irradiated by the incident light beam, so as to generate a large amount of negative and positive charges. The negative changes move toward the second electrode, and the positive charges move toward the first electrode, so as to generate an electric field between the first electrode and the photoelectric conversion layer, and generate a large amount of negative charges at a surface of the droplet, thereby to change the droplet from a hydrophobic state to a hydrophilic state.

According to the method in the embodiments of the present disclosure, the external electric field is applied between the first electrode and the second electrode, and the photoelectric conversion layer is irradiated by the incident light beam, so as to generate the negative and positive charges, generate the electric field between the photoelectric conversion layer and the first electrode, and change the hydrophobic state of the droplet to the hydrophilic state, thereby to drive the droplet to move. The droplet is driven to move by the incident light beam, so it is unnecessary to connect the driving electrode to an external circuit and provide a wire therebetween, thereby it is able to facilitate the design of a large-scale circuit and the manufacture of a large-size digital micro-fluidic chip, and prevent the droplet from being adversely affected by the electrode wire. In addition, it is merely necessary to apply a negative voltage and a positive voltage to the first electrode and the second electrode respectively, so it is unnecessary to provide a complex circuit driving design and a high-voltage power source, thereby to effectively reduce the manufacture coast and simplify the manufacture process.

The present disclosure further provides in some embodiments a micro-fluidic element including the above-mentioned micro-fluidic chip. According to the micro-fluidic element in the embodiments of the present disclosure, the external electric field is applied between the first electrode and the second electrode, and the photoelectric conversion layer is irradiated by the incident light beam, so as to generate the negative and positive charges, generate the electric field between the photoelectric conversion layer and the first electrode, and change the hydrophobic state of the droplet to the hydrophilic state, thereby to drive the droplet to move. The micro-fluidic element may further include such pretreatment members as a droplet generation member, a droplet transportation member, a droplet mixing member and a droplet separation member, as well as relevant driving circuits and a power source, which are known in the art and will not be particularly defined herein.

The present disclosure further provides in some embodiments a biosensor including the above-mentioned micro-fluidic element. The biosensor may further includes relevant driving circuits, a power source, a signal collection member and a signal processing member, which are known in the art and will not be particularly defined herein.

According to the biosensor in the embodiments of the present disclosure, the droplet is driven to move by the incident light beam, it is unnecessary to connect the driving electrode to an external circuit and provide a wire therebetween, thereby it is able to facilitate the design of a large-scale circuit and the manufacture of a large-size digital micro-fluidic chip, and prevent the droplet from being adversely affected by the electrode wire. In addition, it is merely necessary to apply a negative voltage and a positive voltage to the first electrode and the second electrode respectively, so it is unnecessary to provide a complex circuit driving design and a high-voltage power source, thereby to effectively reduce the manufacture coast and simplify the manufacture process.

According to the micro-fluidic chip, its driving method, the micro-fluidic element and the biosensor in the embodiments of the present disclosure, the external electric field is applied between the first electrode and the second electrode, and the photoelectric conversion layer is irradiated by the incident light beam, so as to generate the negative and positive charges, generate the electric field between the photoelectric conversion layer and the first electrode, and change the hydrophobic state of the droplet to the hydrophilic state, thereby to drive the droplet to move. The droplet is driven to move by the incident light beam, so it is unnecessary to connect the driving electrode to an external circuit and provide a wire therebetween, thereby it is able to facilitate the design of a large-scale circuit and the manufacture of a large-size digital micro-fluidic chip, and prevent the droplet from being adversely affected by the electrode wire. In addition, it is merely necessary to apply a negative voltage and a positive voltage to the first electrode and the second electrode respectively, so it is unnecessary to provide a complex circuit driving design and a high-voltage power source, thereby to effectively reduce the manufacture coast and simplify the manufacture process.

The above are merely the preferred embodiments of the present disclosure, but the present disclosure is not limited thereto. Obviously, a person skilled in the art may make further modifications and improvements without departing from the spirit of the present disclosure, and these modifications and improvements shall also fall within the scope of the present disclosure.

What is claimed is:

1. A micro-fluidic chip, comprising a photoelectric conversion layer, a first electrode, and a second electrode arranged opposite to the first electrode, wherein a channel for droplets is arranged between the first electrode and the photoelectric conversion layer, and the photoelectric conversion layer is arranged at a side of the second electrode adjacent to the first electrode and configured to convert an incident light beam into a charge signal to drive the droplets in the channel to move.

2. The micro-fluidic chip according to claim 1, wherein the photoelectric conversion layer is a Positive-Intrinsic-Negative (PIN) photoelectric semiconductor layer.

3. The micro-fluidic chip according to claim 2, wherein the PIN photoelectric semiconductor layer comprises a P-type semiconductor layer, an I-type semiconductor layer and an N-type semiconductor layer laminated one on another, and the N-type semiconductor layer is arranged between the second electrode and the I-type semiconductor layer.

4. The micro-fluidic chip according to claim 3, wherein the P-type semiconductor layer is a P-type amorphous silicon (a-Si) layer, and the I-type semiconductor layer is an I-type a-Si layer, and the N-type semiconductor layer is an N-type a-Si layer.

5. The micro-fluidic chip according to claim 1, further comprising a dielectric layer arranged at a side of the photoelectric conversion layer adjacent to the first electrode, wherein the channel is arranged between the first electrode and the dielectric layer.

6. The micro-fluidic chip according to claim 5, wherein the dielectric layer is made of at least one of silicon nitride, silicon dioxide and ferroelectric copolymer.

7. The micro-fluidic chip according to claim 5, further comprising a first hydrophobic layer arranged at a side of the first electrode adjacent to the second electrode and a second hydrophobic layer arranged at a side of the dielectric layer adjacent to the first electrode, wherein the channel is arranged between the first hydrophobic layer and the second hydrophobic layer.

8. The micro-fluidic chip according to claim 5, further comprising a third electrode arranged between the dielectric layer and the photoelectric conversion layer.

9. The micro-fluidic chip according to claim 8, wherein the third electrode is an electrode array.

10. The micro-fluidic chip according to claim 1, wherein the first electrode is a surface-like electrode, or the second electrode is a surface-like electrode, or the first electrode and the second electrode are both surface-like electrodes.

11. The micro-fluidic chip according to claim 1, further comprising a first substrate arranged at a side of the first electrode away from the second electrode and a second substrate arranged at a side of the second electrode away from the first electrode.

12. A method for driving the micro-fluidic chip according to claim 1, comprising steps of:
applying a voltage between a first electrode and a second electrode; and
converting, by a photoelectric conversion layer, an incident light beam into a charge signal to drive droplets in a channel to move.

13. A micro-fluidic element, comprising the micro-fluidic chip according to claim 1.

14. The micro-fluidic element according to claim 13, wherein the photoelectric conversion layer is a Positive-Intrinsic-Negative (PIN) photoelectric semiconductor layer.

15. The micro-fluidic element according to claim 14, wherein the PIN photoelectric semiconductor layer comprises a P-type semiconductor layer, an I-type semiconductor layer and an N-type semiconductor layer laminated one on another, and the N-type semiconductor layer is arranged between the second electrode and the I-type semiconductor layer.

16. The micro-fluidic element according to claim 15, wherein the P-type semiconductor layer is a P-type amorphous silicon (a-Si) layer, and the I-type semiconductor layer is an I-type a-Si layer, and the N-type semiconductor layer is an N-type a-Si layer.

17. The micro-fluidic element according to claim 13, wherein the micro-fluidic chip further comprises a dielectric layer arranged at a side of the photoelectric conversion layer adjacent to the first electrode, and the channel is arranged between the first electrode and the dielectric layer.

18. The micro-fluidic element according to claim 17, wherein the dielectric layer is made of at least one of silicon nitride, silicon dioxide and ferroelectric copolymer.

19. The micro-fluidic element according to claim 17, wherein the micro-fluidic chip further comprises a first hydrophobic layer arranged at a side of the first electrode adjacent to the second electrode and a second hydrophobic layer arranged at a side of the dielectric layer adjacent to the first electrode, and the channel is arranged between the first hydrophobic layer and the second hydrophobic layer.

20. A biosensor, comprising the micro-fluidic element according to claim 13.
